(19) United States
(12) Patent Application Publication
(10) Pub. No.: US 2014/0148801 A1
(43) Pub. Date: May 29, 2014

(54) SURGICAL DEVICE

(71) Applicant: MEDTRONIC ADVANCED ENERGY LLC, Minneapolis, MN (US)

(72) Inventors: Tony Asher, Charlotte, NC (US); Roger D. Greeley, Portsmouth, NH (US); Anthony R. DePasqua, Newburyport, MA (US)

(73) Assignee: MEDTRONIC ADVANCED ENERGY LLC, Minneapolis, MN (US)

(21) Appl. No.: 14/087,789

(22) Filed: Nov. 22, 2013

Related U.S. Application Data
(60) Provisional application No. 61/729,979, filed on Nov. 26, 2012.

(51) Int. Cl. A61B 18/08; A61B 18/18

(52) U.S. Cl. CPC A61B 18/085; A61B 18/18
USPC 606/33; 606/167; 606/41

(57) ABSTRACT

A surgical device includes a longitudinal first leg having a first distal tip portion pivotably coupled to a longitudinal second leg having a second distal tip portion. The first and second legs are transitionable between a first position such that the first and second tip portions are spaced apart from each other and a second position where the first and second tip portions are proximate each other. The first tip portion includes a concave face and the second tip portion includes a convex face configured such that the first and second faces oppose each other in the first and second positions and touch each other in the second position. The convex face fits within the concave face in the second position.

This Non-Provisional patent application claims the benefit of U.S. Provisional Patent Application Ser. No. 61/729,979, filed Nov. 26, 2012, and titled “BIPOLAR FORCEPS WITH TIPS FOR RF CUTTING AND IRRIGATED COAGULATION,” which is herein incorporated by reference.

### BACKGROUND

Two surgical tasks typically encountered in both open and endoscopic surgery include cutting or severing tissues and controlling bleeding. A large percentage of the time spent during surgery involves the control of bleeding. If bleeding is not controlled, blood can obscure the view of the surgical space on a patient, adversely affect the precision of the cutting or severing tissue, and prolong the surgery. Too much blood loss can cause trauma to the patient that may require a blood transfusion.

Cutting or ligating blood vessels include special considerations. Surgeons often have difficulty suturing vessels. Other traditional methods for controlling bleeding such as clamping or tying-off transected blood vessels are made difficult if surgery is performed in a remote operating space. Traditional methods also impractically prolong surgeries in cases where many blood vessels are cut. For example, a surgeon attempting to resect a neoplasm such as a brain tumor or spinal tumor may transect hundreds of blood vessels before the abnormal mass can be removed.

Electrosurgical instruments are often used to control bleeding in such circumstances. Electrosurgical forceps and hemostats can be used to cauterize, coagulate/desiccate or simply reduce blood flow by controlling electrosurgical energy applied to the tissue. Small blood vessels, e.g., those having a diameter of less than about two millimeters, can be coapted through coagulation, i.e., the process of desiccating tissue where the tissue cells are ruptured and dried. Larger blood vessels may be coapted through sealing, i.e., the process of liquefying the collagen in the tissue so that it reforms into a fused mass. A surgeon can then cut through the coapted portion of the vessel with a device such as microscissors to effect little or no bleeding in the surgical space.

In order to resect a neoplasm in the brain or spinal column, a surgeon performs the surgical tasks of controlling the bleeding and cutting the tissues with multiple surgical instruments. Switching between instruments is time consuming, tedious, and could cause the surgeon to lose focus. Difficulties lie in finding the coapted portions after the surgeon has switch to a cutting device in the case of a surgeon using multiple devices and cutting through the coapted portions so that both ends of the ligated vessels remain coapted.

### SUMMARY

This summary is provided to introduce a selection of concepts in a simplified form that are further described below in the Detailed Description. This summary is not intended to identify key features or essential features of the claimed subject matter, nor is it intended to limit the scope of the claimed subject matter.

In one aspect, the disclosure relates to a surgical device. The surgical device includes a longitudinal first leg having a first distal tip portion pivotably coupled to a longitudinal second leg having a second distal tip portion. The first and second legs are transitionable between a first position where the first and second tip portions are spaced apart from each other and a second position where the first and second tip portions are proximate each other. The first tip portion includes a concave face and the second tip portion includes a convex face configured such that the first and second faces oppose each other in the first and second positions and touch each other in the second position. The convex face fits within the concave face in the second position.

In another aspect, the disclosure relates to an electrosurgical device. The electrosurgical device includes a longitudinal first leg having a first distal electrode pivotably coupled to a longitudinal second leg having a second distal electrode. The first and second legs are transitionable from a first position where that the first and second electrodes are spaced apart from each other to a second position where the first and second electrodes are proximate each other. The first electrode includes a concave face and the second electrode includes a convex face such that the first and second faces oppose each other in the first and second positions and touch each other in the second position. The convex face fits within the concave face in the second position.

In another aspect, the disclosure related to a bipolar electrosurgical forceps. The bipolar forceps include a longitudinal first leg having a first distal tip, where the first distal tip includes a first electrode and a first irrigational tube. The bipolar forceps also include a longitudinal second leg having a second distal tip, where the second distal tip includes a second electrode and a second irrigational tube. The first leg is pivotably coupled to the second leg, and the first and second legs are yieldably urged apart in a first position where that the first and second tips are spaced apart from each other and transitionable under force to a second position where the first and second tips are proximate each other. The first electrode includes a concave face and the second electrode includes a convex face such that the first and second faces oppose each other in the first and second positions and touch each other in the second position. The convex face fits within the concave face in the second position.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated, as they become better understood by reference to the following detailed description. The elements of the drawings are not necessarily to scale relative to each other. Like reference numerals designate corresponding similar parts.

FIG. 1 is a schematic view illustrating an electrosurgical system including an electrosurgical device for coapting and cutting according to the present description.

FIG. 2 is a schematic view illustrating an example of the electrosurgical device of FIG. 1.

FIG. 3A is a schematic view illustrating a portion of the electrosurgical device of FIG. 1.

FIG. 3B is a schematic view illustrating another example of a portion of the electrosurgical device of FIG. 1

FIGS. 4A and 4B are schematic views illustrating a feature of the example electrosurgical device of FIG. 3.

FIGS. 5A, 5B, and 5C are schematic views illustrating alternative features of the example electrosurgical device of FIG. 3.

FIGS. 6A and 6B are schematic views illustrating a process for using the example electrosurgical device of FIG. 3.

FIG. 7 is a graph illustrating a feature used in the process of FIG. 6A.

### DETAILED DESCRIPTION

In the following Detailed Description, reference is made to the accompanying drawings, which form a part hereof, and in which is shown by way of illustration specific embodiments, or examples, in which the disclosure may be practiced. Other embodiments may be utilized and structural or logical changes may be made without departing from the scope of the present disclosure. The following Detailed Description, therefore, is not to be taken in a limiting sense, and the scope of the present disclosure is defined by the appended claims. It is to be understood that features of the described various exemplary embodiments may be combined with each other, unless specifically noted otherwise.

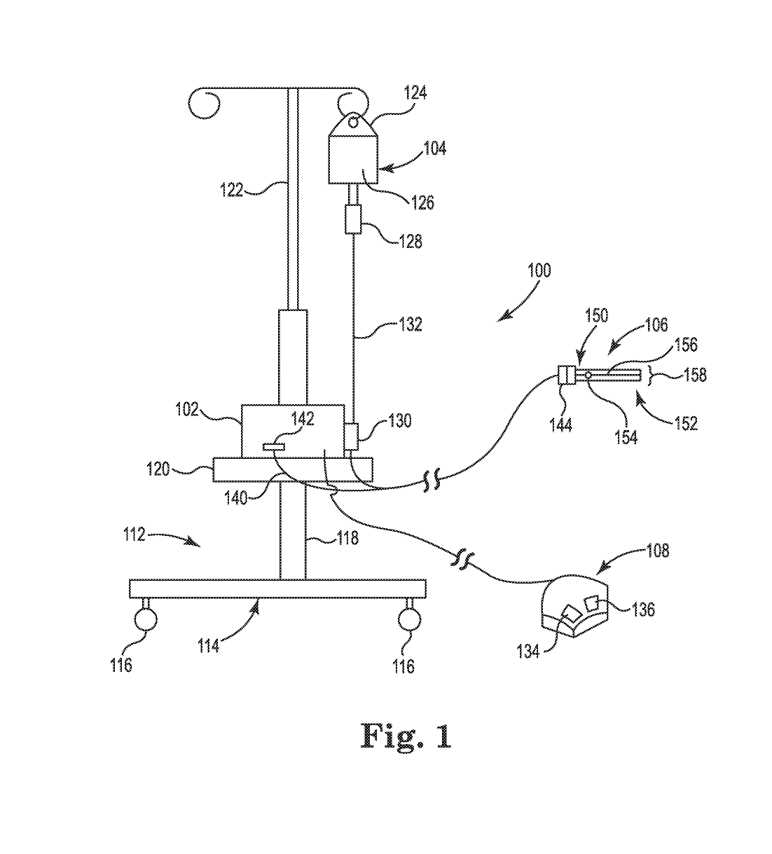

FIG. 1 illustrates an electrosurgical system 100 having an electrosurgical unit 102 coupled to a fluid source 104 and an electrosurgical device 106, activated with a switch 108, and plugged into an electrical power source (not shown). Unit 102 in the example is on a movable cart 112 having a chassis 114. Chassis 114 includes wheels 116 and a support member 118 having a shelf 120 and pole 122.

In one example, the electrosurgical unit 102 can be a generator such as the generator sold under the trade designation Aquamantys 3, available from Medtronic, Inc., of Minnesota, United States. The electrosurgical unit 102 in the example is configured to provide both electrical energy and fluid to the electrosurgical device 106 in one of two or more selectable and mutually exclusive modes. In a first mode, the unit 102 provides both fluid and a first selected amount of electrical energy to the device 106 that is suitable to irrigate and to coapt tissue such as blood vessels. In a second mode, the unit provides a second selected amount of electrical energy to the device that is suitable to cut the tissue, such as the coapted tissue. In one example, the mode settings of electrical energy and fluid flow are preselected prior to surgery. The modes are selected and controlled with switch 108. Examples of suitable generator and flow rate controllers are described in U.S. Pat. No. 7,815,634, and published U.S. Pat. Application Nos. 2001-0032002; 2006-0149225 and 2005-0090816, which are incorporated by reference into this disclosure.

Fluid source 104, in one example, includes a bag 124 of fluid 126 hung from pole 122. Fluid 126 flows from the bag 124 to a drip chamber 128. Flexible delivery tubing 130 carries the fluid to from the drip chamber 128 to the electrosurgical device 106. In one example, the fluid 126 includes saline and can include physiologic saline. Saline is an electrically conductive fluid, and other suitable electrically conductive fluids can be used. In other examples, the fluid 126 may include a nonconductive fluid, such as deionized water, which may still provide advantages over using no fluid.

A pump, such as pump 130, conveys fluid 126 to the electrosurgical device 106, and unit 102 can provide controls for the fluid flow. In one example, delivery tubing 132 is passed through pump 130 on unit 102. Pump 130 in one example is a peristaltic pump such as a rotary peristaltic pump or a linear peristaltic pump. Pump 130 can convey the fluid 126 through the delivery tubing 132 by way of intermittent forces placed on the external surface of the delivery tubing 132. Peristaltic pumps are often preferred because the mechanical elements of the pump places forces are placed on the external surface of the delivery tubing and do not come into direct contact with the fluid, which can reduce the likelihood of fluid contamination. Other examples of the electrosurgical instrument 100 might not include a pump, and fluid 126 is provided to the electrosurgical device 106 via gravity.

Switch 108 can be used to activate or control fluid flow to the electrosurgical device 106. In one example, the switch can include two activators 134, 136 or toggles. One activator can be depressed to select the first mode to coapt the tissue and a second activator can be depressed to select the second mode to cut the tissue. The switch 108 can be a foot switch, which may be preferable over a finger switch or other switch due to provide more stability and steadiness at the electrosurgical device 106, for example, when used in neurosurgery.

The electrosurgical device 106 is also in electrical communication with the electro surgical unit 102, for example, via cable 140 that can include electrically insulated wire conductors, a plug 142 at the electrosurgical unit 106, and another plug 144 to fit the electrosurgical unit 102. The electrosurgical unit 102 provides electrical energy via cable 140 to the electrosurgical device 106, such as radio-frequency (RF) energy. In one example, the delivery tubing 132 can be coupled to the cable 140 over a portion of its length in a way so that keep neat the connections between the electrosurgical unit 102 and the electrosurgical device 106.

The electrosurgical device 106 includes a proximal portion 150, distal portion 152, and a pivot 154. The device 106 is configured in the form similar to tweezers if the pivot 154 is disposed at the proximal portion 150, and device 106 is configured similar to form of scissors, or microscissors, if the pivot 154 is disposed between the proximal portion 150 and the distal portion 152. In either case, the device 106 is transitional between a first or open position where the distal portions are spaced apart from and a second or closed position where the distal portions are proximate to each other. Device 106 can be of any suitable length. The proximal portion 150 is configured to receive plug 142. The device 106 includes longitudinally extending legs 156 with tips 158, or blades, designed to engage tissue at the distal portion 152. The tips 158 include one or more electrodes in electrical communication with the electrosurgical unit 102 to provide energy to the tissue in each of the coapting and cutting modes. The tips 158 also include perforated tubing in fluid communication with the fluid source 104 to irrigate the tissue with fluid 126 in the coapting mode.

The electrosurgical device 106 can take a number of configurations. For instance, the device 106 can be reusable after it is cleaned and disinfected or sterilized before each use or it can be disposable after each use. Each type can include a bipolar variation with two operating electrodes or a monopolar variation with one operating electrode. Radio frequency energy is passed between the two closely spaced electrodes in bipolar instruments. In monopolar instruments, RF energy is conducted through the operating electrode to a remote conductive body-plate or grounding pad. Many neurosurgeons prefer bipolar instruments that causes the energy and current flow remains localized to the tissue adjacent to the working area between the electrodes rather than a monopolar instrument that causes energy and current to flow through brain or spinal tissue to the grounding pad. U.S. Pat. No. 8,414,572 and published U.S. Pat. No. 2012-0004657 describe examples of devices operable in the monopolar and bipolar modes and are incorporated by reference into this disclosure.

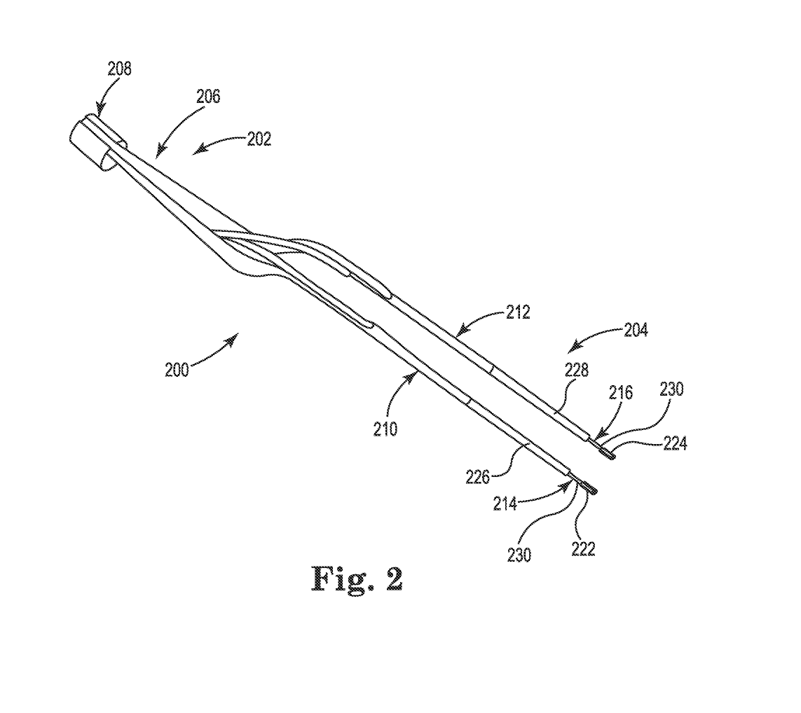

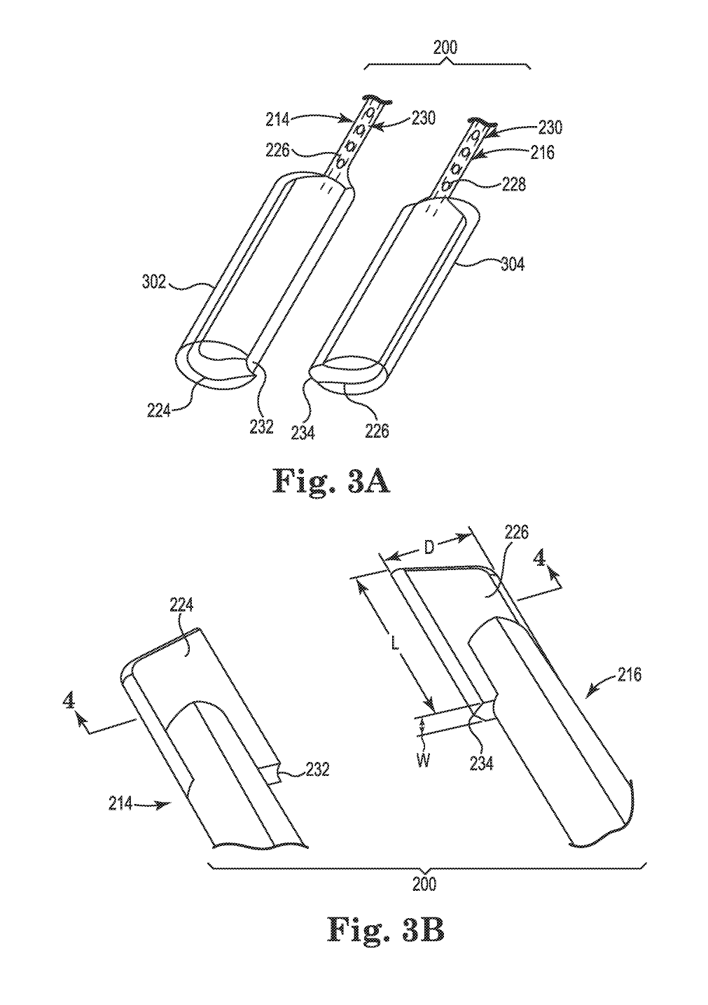

FIGS. 2, 3A and 3B illustrate an example electrosurgical device 106 configured in the form a forceps 200. The forceps 200 includes a proximal portion 202 and a distal portion 204, corresponding with the proximal portion 150 and the distal portion 152, respectively, of the electrosurgical device 106. The proximal portion 202 in the example includes a hinge 206, corresponding with pivot 154, and a plug 208 configured to connect with plug 142 to be in electrical and fluid communication with the electrosurgical unit 102. The distal portion 204 includes two legs 210, 212 each having tips 214, 216, respectively, designed for engaging tissue. The tips 214, 216 can be configured in as the ends of common tweezers. In the illustrated example, however, the tips 214, 216 are bayonet shaped. The bayonet shaped tips 214, 216 allow for greater visibility of the tissue to be treated. Other shapes of the tips 214, 216 are contemplated. In one example, the forceps 200 are at least partially made from high-grade carbon steel that can withstand repeated sterilization in high temperature autoclaves or other suitable medical grade material.

The hinge can be configured to allow the tips to move freely or to be held in a particular position. In one example, the hinge 206 is formed from fusing the legs 210, 212 together at the proximal portion 202. In another example, hinge 206 is integrally formed with the legs 210, 212. Using these or similar examples of hinges, spring tension from the hinge 206 holds the tips 214, 216 apart in an open position until a force is applied to bring the tips together in a closed position.

In the example of forceps 200, tips 214, 216 include electrodes 222, 224, respectively, in electrical communication with plug 208 and irrigation ducts 226, 228, respectively, within the tips 214, 216, in fluid communication with plug 208. Each of the irrigation ducts 226, 228, are terminated with holes 230 on or proximate to the electrodes 222, 224 to emit or weep fluid 126 from the irrigation ducts 226, 228 during coapting mode. The number of holes 230 can vary considerably, such as from one to ten and, preferably, from two to eight holes 230 on each tip 214, 216. The size of the holes 230 may vary. In some examples, one or both of the tips 214, 216 are associated with porous or microporous material to weep the fluid 126. In the example shown, both tips 214 and 216 include irrigation ducts, but examples are contemplated where irrigation ducts are provided in just one of the tips 214, 216.

The electrodes 222, 224 afford proper application of both coapting energy for coagulation and cutting energy. The electrodes 222, 224 each include opposing faces 232, 234, respectively, or edges that are designed to contact each other in the closed position or to tissue contact the tissue when a force is applied to the legs 210, 212. In the example shown in FIG. 3A, the electrodes 222, 224 are covered with an insulator 302 and 304, respectively, such as glass. In one example, the electrodes 222, 224, are thinner at the opposing faces 232, 234 and are broader on sides 236, 238 away from the tissue forming longitudinal blade electrodes. The opposing faces 232, 234 create a focus for the RF energy that promotes the initiation of plasma to be used in cutting the tissue. The thin opposing faces 232, 234 also can easily pass through tissue like a knife or a scissors blade. The thin aspect of the opposing faces, however, is not ideal in spreading energy to achieve coapting through coagulation. The introduction of fluid 126 from holes 230 disperses the focused RF energy to prevent the creation of plasma, cool the tips 214, 216, couples the RF energy to the tissue, and reduces the amount the electrodes 224, 226 stick to the tissue, which provides for robust coapting through coagulation.

While the thin opposing faces presents advantages, thin opposing faces, such as bayonet shaped tips, can also provide performance drawbacks not addressed with irrigation. For example, as the opposing faces come together in a closed position, they have a tendency to slip off each other if additional force is applied. The opposing faces can be pushed past the closed position and begin become further separated. This effect can be described as “scissoring” where the opposing faces deflect each other and extend past each other. Scissoring can have adverse effects on the tissue in that the field of the RF energy becomes larger than if the opposing faces were more proximate and approaching the closed position. As the field of the RF energy increases, more tissue is affected, and the cutting and coapting of tissue becomes imprecise. A surgeon can find it difficult to detect whether scissoring has occurred at the tips of the forceps, and the surgeon can also find it difficult to determine the precise amount of force to apply to the legs approach the closed position and to avoid scissoring.

FIG. 3B illustrates the electrodes 224, 226, without the insulator 302, 304, in FIG. 3A, to highlight features designed to address the issue of scissoring. Electrode 224 includes a concave configuration to opposing face 232, and electrode 226 includes a convex configuration to opposing face 234. When the electrodes are in a closed position, the convex opposing face 232 fits within the concave opposing face 234 to hold it in place and reduce the tendency of scissoring. The “self-centering” feature of the concave and convex faces is present when there is no tissue present and the self-centering feature is also present when there is tissue disposed between the opposing faces 232, 234. For purposes of illustration, dimension L is a length of electrode 226, dimension D is a depth of electrode 226, and dimension W is a width of the opposing face 234. Similar dimensions can be used to describe the features of electrode 224. In one example, the length L is in the range of 0.2 to 0.4 inches, the depth D is in the range of 0.02 to 0.12 inches, and the width W of the opposing face 234 is in the range of 0.01 to 0.04 inches. Similar dimensions can be applied to electrode 224 and opposing face 232.

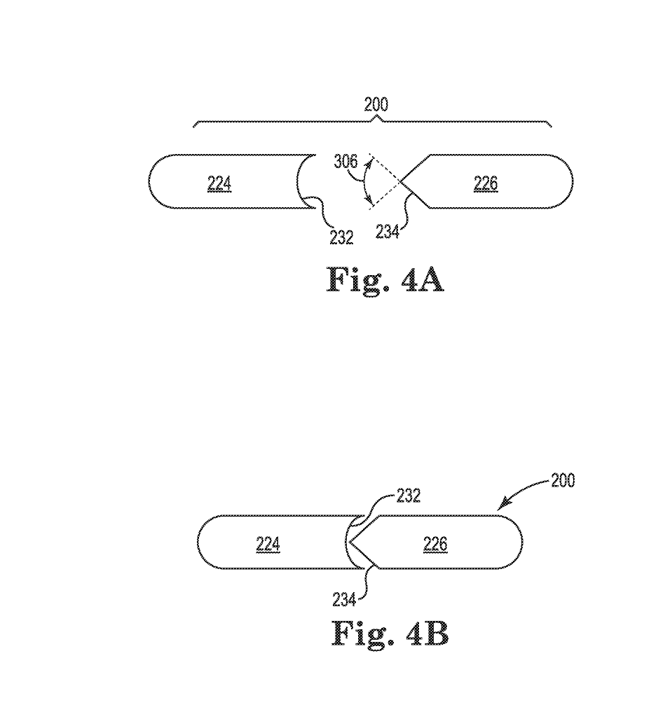

FIGS. 4A and 4B illustrates features of the electrodes designed to address the issue of scissoring in a cross section view of electrodes 224, 226 from FIG. 3B taken along lines 4-4. FIG. 4A illustrates electrodes 224, 226 in an open position with opposing faces 232, 234 spaced-apart from each other. Electrode 224 includes a concave configuration to opposing face 232, and electrode 226 includes a convex configuration to opposing face 234. In one example, the concave face 232 includes a radius in the range of 0.01 inches to 0.05 inches. In one example, the convex face 232 is constructed to include a taper or V-shaped face having an angle 306 in the range of 30 degrees to 60 degrees. FIG. 4B illustrates electrodes 224, 226 in a closed position with opposing faces 232, 234 touching each other. The concave and convex opposing faces extend along the length of the electrodes 224, 226. When the electrodes 224, 226 are placed in the closed position, the convex opposing face 234 fits within concave opposing face 232. For example, the entire convex face 234 can fit within, i.e., be disposed within, the concave face 232 or a portion of the convex face 234, such as a leading tip of the convex face, can fit within the concave face 232. The concave opposing face 234 hold the convex opposing face so that additional force does not deflect the opposing faces 232, 234 and cause scissoring.

The range of dimensions described with reference to FIGS. 3B and 4A are particularly suited for microsurgery, such as brain or spinal column surgery, with particular concerns of precision, heat transfer, and the like. Larger forceps 200 are not as advantageous for coapting and cutting blood vessels in brain and spinal column surgery and particularly for removing neoplasms in these tissues because they are imprecise in coapting and cutting the vast amount of small blood vessels in these regions and introduce to much heat and RF energy, which can cause permanent damage to such sensitive tissue. Further, smaller forceps, or forceps that are closed with lower amounts of force in general, are more prone to the problem of scissoring.

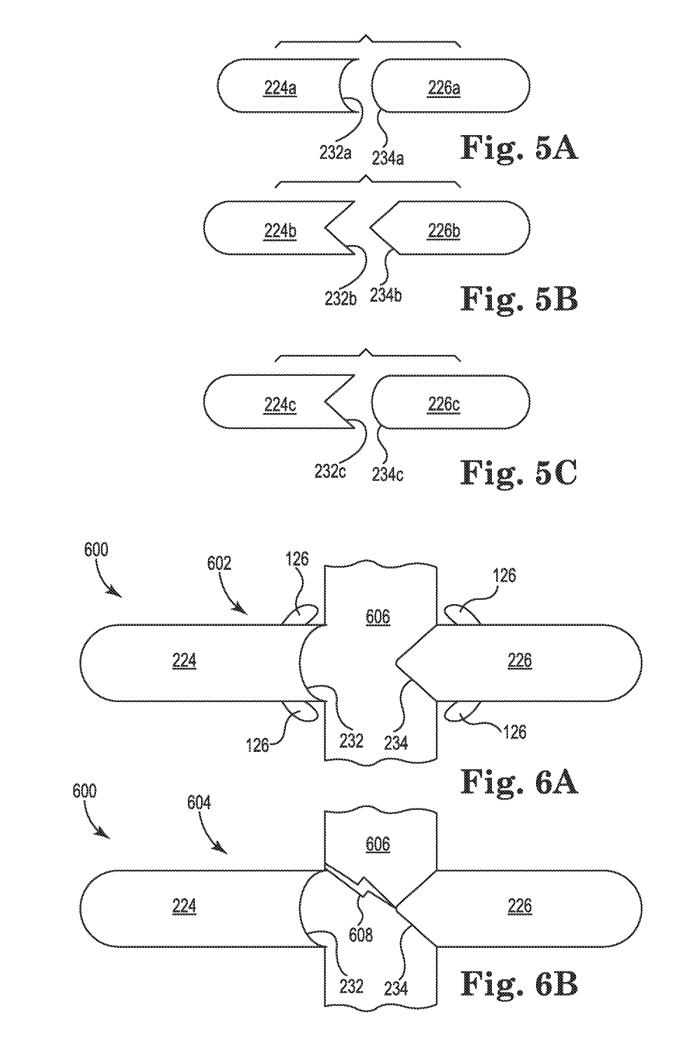

Although the electrodes 224, 226 are shown having opposing faces 232, 234 particularly design as mating concave and convex semicircles, other concave and convex configurations are possible. FIGS. 5A to 5C illustrates some examples of suitable concave and convex opposing faces also designed to resist scissoring. FIG. 5A illustrates electrodes 224a, 226a having a generally curvilinear, such as U-shaped, concave/convex opposing faces 232a, 234a. FIG. 5B illustrates electrodes 224b, 226b having V-shaped concave/convex opposing faces 232b, 234b. FIG. 5A illustrates electrodes 224c, 226c having mismatched shaped concave/convex opposing faces such as a V-shaped concave face 232c and a U-shaped convex face 234c. Other configurations are contemplated. The concave/convex faces can include a particular shape to the cross section, as illustrated in FIGS. 4A and 5A to 5C, or can just be a machined detent in one of the opposing faces that is wider than the other opposing face.

The electrodes 224, 226 can be formed from a metal such as titanium, copper, tantalum, molybdenum, tungsten, or stainless steel. In some variations, the electrodes are formed from a metal foil. These materials are capable of withstanding the high temperatures used in the creation of plasma in the cutting mode. Additionally, these materials have sufficient rigidity when used as a blade. Further, these materials have a low thermal conductivity, which reduces the outflow heat from electrode to the tissue. In one example, the electrodes 224, 226 are machined so the opposing faces 232, 234 are approximately 500 micrometers wide.

One or more of the electrodes can also be covered with one or more insulators 302, 304, as illustrated in FIG. 3A, in such a manner as to leave the opposing faces 232, 234 substantially exposed or substantially not covered with insulation. Additionally, the distal tip of the electrodes 224, 226 can be substantially exposed or substantially not covered with insulation. An insulator can include glass, ceramic, glass enamel, or other suitable electrode insulator. The presence of insulation on the electrodes further helps to focus the RF energy to the opposing faces and also imparts less heating to the tissue adjacent the insulated portions of the electrode. Excessive thermal damage has the potential to result in necrosis. In one example, the insulator is affixed to the electrodes and does not cover the opposing faces 232, 234, or the insulator is later machined off of the opposing faces 232, 234 or eroded with an electric pulse sent through the electrodes 224, 226.

FIGS. 6A and 6B illustrate a process 600 of coapting 602 and cutting 604 tissues with the electrosurgical system 100 using electrosurgical device 106 such as forceps 200. The electrodes 224, 226 are positioned on either side of the tissue 606. Generator, such as unit 102, provides a RF waveform to the electrodes operating in a bipolar mode. In one example, the waveform can be a monopolar waveform to one of the electrodes, such as electrode 224, with electrode 226 acting as the return electrode to confine the RF field to the tissue adjacent the electrodes. Pump 130 provides fluid 126 to the forceps 200. Example waveforms are described in U.S. Pat. No. 8,323,276 and the above-identified U.S. Published patent applications, which are incorporated by reference into this disclosure.

The surgeon selects the coapting mode or the cutting mode with switch 108. The generator is preconfigured to deliver the first selected power or the second selected power to the electrosurgical device 106 depending on which mode is selected. If the coapting mode is selected, the first selected power is delivered to one of the electrodes 224, 226 and the pump 130 is turned on to provide fluid to the tips 214, 216 in the example. If the cutting mode is selected, the second selected power is delivered to one of the electrodes 224, 226 and the pump 130 is turned off so fluid 126 is not supplied to the tips 214, 216.

In one example, the tissue 604 can include small blood vessels in the brain or spine of a patient. Electrosurgical devices 106 like forceps 200 or microscissors engage the tissue 604 with small electrodes to minimize disruption to adjacent tissues and to improve visibility at the surgical site. The small electrodes also allow for precise coapting and cutting with the same device. The self-alignment provide by the concave/convex opposing faces 232, 234 improves precision in coapting and cutting as the surgeon can grasp the tissue 604 with sufficient force that would otherwise deflect or scissors other small electrodes.

In coapting mode, shown in FIG. 6A, the first selected power and fluid are delivered to the tissue 604, which will coagulate small blood vessels. The tissue and electrodes 224, 226 are irrigated with fluid 126 to disperse the RF energy. The presence of the fluid 126 along with the RF energy reduces current concentration at the opposing faces 232, 234, which can prevent the creation of plasma at the tissue 604. The fluid 126 also couples the RF energy to the tissue 604 and cools the electrodes 224, 226. The RF energy heats the tissue 604 and coagulates the blood vessel.

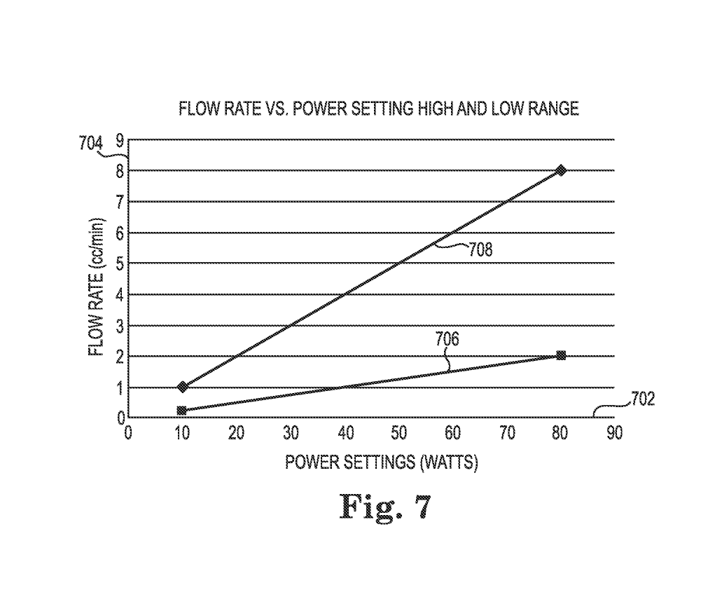

FIG. 7 is a graph illustrating a relationship of power setting 702 in Watts to flow rate 704 in cubic centimeters per minute. Line 706 indicates a preferred low range of flow to power, and line 708 indicates a preferred high range of flow to power. The amount of the first selected power used to coagulate blood is preferably in the range of 10 W to 50 W, with a preferred corresponding flow rate indicated between lines 706 and 708 for a given power setting. In one example, an alternating RF waveform of approximately 25 W, as the first selected power, works well to coagulate blood vessels in soft tissue such as brain tissue.

In cutting mode, shown in FIG. 6B, a second selected power without irrigating fluid is applied to the coagulated tissue 604 to cut the tissue with no or reduced bleeding. The RF energy is concentrated in the opposing faces and creates plasma, indicated schematically at 608, that is used to cut through the tissue. The amount of the second selected power is preferably in the range of 10 W to 30 W for blood vessels in the brain and spinal column. In one example, an alternating RF waveform of approximately 15 W, as the second selected power, works well with blood vessels in the brain and spinal column.

Although the present disclosure has been described with reference to preferred embodiments, workers skilled in the art will recognize that changes can be made in form and detail without departing from the spirit and scope of the present disclosure.

What is claimed is:

1. An surgical device, comprising:
a longitudinal first leg having a first distal tip portion;
a longitudinal second leg having a second distal tip portion, the first leg pivotably coupled to the second leg, wherein the first and second legs are transitionable between a first position such that the first and second tip portions are spaced apart from each other and a second position where the first and second tip portions are proximate each other;
wherein the first tip portion includes a concave face and the second tip portion includes a convex face such that the first and second faces oppose each other in the first and second positions and touch each other in the second position;
wherein the convex face fits within the concave face in the second position.

2. The surgical device of claim 1, wherein the first leg includes a first proximal portion and the second leg includes a second proximal portion, and the first proximal portion is coupled to the second proximal portion to form a hinge.

3. The surgical device of claim 2, wherein the first proximal portion is fused to the second proximal portion.

4. The surgical device of claim 2, wherein the first and second legs are yieldably urged apart in the first position and transitionable under force to a second position.

5. The surgical device of claim 1, wherein the first tip portion includes first longitudinal length and the concave face extends along the first longitudinal length.

6. The surgical device of claim 5, wherein the second tip portion includes a second longitudinal length and the convex face extend along the second longitudinal length.

7. The surgical device of claim 1 wherein the first tip portion is narrowest at the first opposing face.

8. The surgical device of claim 1, wherein the first and second tip portions are configured to cut tissue.

9. The surgical device of claim 1 comprises carbon steel.

10. An electrosurgical device, comprising:
a longitudinal first leg having a first distal electrode;
a longitudinal second leg having a second distal electrode, the first leg pivotably coupled to the second leg, wherein the first and second legs are transitionable from a first position such that the first and second electrodes are spaced apart from each other to a second position where the first and second electrodes are proximate each other;
wherein the first electrode includes a concave face and the second electrode includes a convex face such that the first and second faces oppose each other in the first and second positions and touch each other in the second position;
wherein the convex face fits within the concave face in the second position.

11. The electrosurgical device of claim 10, wherein the electrodes are constructed from titanium, tantalum, molybdenum, tungsten, or stainless steel.

12. The electrosurgical device of claim 10, wherein the at least one of the first and second electrodes is covered with an insulator.

13. The electrosurgical device of claim 12, wherein the first and second electrodes are at least partially covered in an insulator.

14. The electrosurgical device of claim 12, wherein the insulator is glass.

15. The electrosurgical device of claim 12, wherein the at least one electrode covered in the insulator includes is substantially exposed at the opposing face.

16. The electrosurgical device of claim 10, wherein the convex face is generally V-shaped and the concave face is generally curvilinear.

17. The electrosurgical device of claim 10, wherein the electrodes are configured to focus radiofrequency energy at the opposing faces.

18. A bipolar electrosurgical forceps, comprising:
a longitudinal first leg having a first distal tip, wherein the first distal tip includes a first electrode and a first irrigational tube;
a longitudinal second leg having a second distal tip, wherein the second distal tip includes a second electrode and a second irrigational tube;
wherein the first leg is pivotably coupled to the second leg, and wherein the first and second legs are yieldably urged apart in a first position such that the first and second tips are spaced apart from each other and transitionable under force to a second position where the first and second tips are proximate each other;
wherein the first electrode include a concave face and the second electrode includes a convex face such that the first and second faces oppose each other in the first and second positions and touch each other in the second position;
wherein the convex face fits within the concave face in the second position.

19. The bipolar electrosurgical forceps of claim 18, and comprising a plug in fluid communication with the first and second irrigation tubes and in electrical communication with the first and second electrodes.

20. The bipolar electrosurgical forceps of claim 18 wherein the first and second irrigation ducts are terminated with holes configure to emit fluid proximate the first and second electrodes.
